SEQUENCE LISTING (1 sequence)

SEQ ID NO: 1 - protein, 546 aa, Varicella Zoster
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

(19) United States
(12) Patent Application Publication
(10) Pub. No.: US 2021/0187098 A1
(43) Pub. Date: Jun. 24, 2021

(54) VACCINATION

(71) Applicant: GLAXOSMITHKLINE BIOLOGICALS SA, Rixensart (BE)

(72) Inventor: Cornelia OOSTVOGELS, Wavre (BE)

(73) Assignee: GLAXOSMITHKLINE BIOLOGICALS SA, Rixensart (BE)

(21) Appl. No.: 16/607,845

(22) PCT Filed: Apr. 27, 2018
(86) PCT No.: PCT/IB2018/052937

§ 371 Date: Oct. 24, 2019

(30) Foreign Application Priority Data
Apr. 28, 2017 (GB) 1706798.4
May 15, 2017 (GB) 1707785.0

(51) Int. Cl. A61K 39/25

(52) U.S. Cl. CPC A61K 39/25; A61K 2039/55555

(57) ABSTRACT

Immunogenic compositions for use in and methods for protecting against Herpes Zoster (shingles).

### SEQUENCE LISTING

The instant application is filed with an electronically submitted Sequence Listing in ASCII text file format (Name: VB66318_US_ST25.txt; Size: 5 KB; and Date of Creation: 12 Mar. 2020) which is hereby incorporated by reference in its entirety.

### TECHNICAL FIELD

The present invention relates to methods for inducing early protection against and prevention of Herpes Zoster or post herpetic neuralgia, in particular in elderly and immunocompromised human patients.

### BACKGROUND

Herpes Zoster (HZ), also known as shingles, is a common and often debilitating disease that occurs primarily in older or immunocompromised individuals. HZ is caused by the symptomatic reactivation of latent varicella zoster virus (VZV) in the dorsal root and cranial ganglia. The virus is usually acquired during childhood as chickenpox.

The only vaccine currently available with demonstrated efficacy against HZ or post herpetic neuralgia (PHN) is a live attenuated vaccine of VZV OKA strain, marketed as ZOSTAVAX. In the overall population (≥60 YOA), ZOSTAVAX reduced the incidence of HZ by 51.3% (p-value <0.001), although its effectiveness decreased with the age of the vaccinee. In particular, vaccine efficacy (VE) diminished to 37.6% among persons in older age groups (≥70 years of age). ZOSTAVAX is contraindicated in persons with immunodeficiency due to malignancy, human immunodeficiency virus (HIV) infection or immunosuppressive medical therapy. (ZOSTAVAX EMA SPC 2012; Oxman et al. N Engl J Med 2005; 352:2271-2284; Schmader et al. Clin. Infect. Diseases 2012 April; 54(7):922-8). Morrison V. A. et al. reported on the decline in efficacy of ZOSTAVAX becoming increasingly limited beyond 5-8 years post-vaccination and to be no longer statistically significant beyond 8 years (Morrison et al. Clin. Infect. Diseases advance access publication Nov. 20, 2014).

An adjuvanted subunit VZV immunogenic composition is described in WO2006/094756 (U.S. Pat. No. 7,939,084, which is incorporated herein by reference for defining the immunogenic composition). Leroux-Roels I. et al. (J. Infect. Diseases 2012:206 1280-1290) report on a phase I/II clinical trial of the adjuvanted VZV gE subunit vaccine evaluating safety and immunogenicity. The adjuvanted subunit VZV vaccine has shown to provide high efficacy following a 2-dose schedule (Himal L. et al. 2015 NEJM 372(22):2087).

### SUMMARY OF THE INVENTION

The present invention relates to immunogenic compositions for use in and methods for protecting against HZ upon administration of one dose of the composition, and particularly within a short time frame following the administration of the one dose.

The invention also relates to a method for protecting against, preventing, or reducing the incidence of herpes zoster and/or post herpetic neuralgia in an individual comprising the steps of:
a. selecting a subject from a population that is in need of protection against, preventing, or reducing the incidence of herpes zoster and/or post herpetic neuralgia within a limited term after administration, and,
b. administering a single or a first dose of an immunogenic, e.g. vaccine, composition comprising a VZV gE antigen truncated to remove the carboxy terminal anchor region, in combination with an adjuvant comprising a saponin, a TLR-4 agonist and liposomes.

The invention also relates to a method for protecting against, preventing, or reducing the incidence of herpes zoster and/or post herpetic neuralgia in an individual comprising the steps of:
a. selecting a subject from a population that is in need of protection against, preventing, or reducing the incidence of herpes zoster and/or post herpetic neuralgia prior to immunosuppressing therapy, and,
b. administering a single or a first dose of an immunogenic, e.g. vaccine, composition comprising a VZV gE antigen truncated to remove the carboxy terminal anchor region, in combination with an adjuvant comprising a saponin, a TLR-4 agonist and liposomes, where the administration occurs prior to or concomitant with the start of immunosuppressive therapy,

### DESCRIPTION OF THE DRAWINGS

FIG. 1A-1C graph cell-mediated immune response following a 0.2 month 2-dose vaccination schedule with adjuvanted VZV gE (with varying amounts of gE per dose i.e. 25 μg, 50 μg and 100 μg), a single dose of adjuvanted VZV gE (saline administered as first dose followed by adjuvanted VZV gE 100 μg as second dose), or a 0.2 month 2-dose vaccination schedule with VZV gE (100 μg per dose), in the overall cohort of study subjects (FIG. 1A), the cohort of subjects from 60 to 69 years of age (FIG. 1B), and the cohort of subjects greater than or equal to 70 years of age (FIG. 1C). Y-axis is CD4²⁺ T cells/10⁶ CD4⁺ T cells and the X-axis is months following initial vaccination

FIG. 2A-2C graphs VZV gE antibody levels following a 0.2 month 2-dose vaccination schedule with adjuvanted VZV gE, a single dose of adjuvanted VZV gE (with varying amounts of gE per dose i.e. 25 μg, 50 μg and 100 μg), a single dose of adjuvanted VZV gE (saline administered as first dose followed by adjuvanted VZV gE 100 μg as second dose), or a 0.2 month 2-close vaccination schedule with VZV gE (100 μg per dose), in the overall cohort of study subjects (FIG. 2A), the cohort of subjects from 60 to 69 years of age (FIG. 2B), and the cohort of subjects greater than or equal to 70 years of age (FIG. 2C). Y-axis is Antibody Geometric Mean Concentration (GMC) and the X-axis is months following initial vaccination.

FIG. 3A-3B provides the study design of the phase III clinical vaccination trial (Trial I) described in Example 1.

FIG. 4 provides the study design of the phase III clinical vaccination trial (Trial II) described in Example 2.

FIG. 5 provides the study design of the phase III clinical vaccination trial described in Example 3.

FIG. 6 represents immunogenicity data as reported for Example 3: panel A—Humoral immune response to VZV gE/AS01B vaccination: anti-glycoprotein E (gE) antibody concentrations were determined by enzyme-linked immunosorbent assay, geometric mean concentrations (GMCs [mIU/mL]) are reported and error bars indicate 95% confidence intervals (CIs); panel B—cellular immune response to VZV gE/AS01B vaccination, gE-specific CD4+ cells expressing at least 2 activation markers (CD4²⁺) determined by intracellular staining and flow cytometry are reported (data are median cell counts per 10⁶ total peripheral blood mononuclear cells); light bars indicate HZ-PreVac group, dark bars indicate HZ-NonVac group. HZ-NonVac=participants who never received the live-attenuated zoster vaccine (ZVL); HZ-PreVac=participants who received ZVL≥5 years prior to study start.

FIG. 7 provides the design of the clinical trial to assess the immunogenicity and safety of the HZ/su vaccine in adults with solid tumors, vaccinated either before or at the start of immunosuppressive chemotherapy (chemo).

FIG. 8 graphs GMC of anti-gE antibodies (adapted ATP cohort for humoral immunogenicity) in ST subjects, where gE=glycoprotein E; ATP=according-to-protocol; GMC=geometric mean concentration; IU=international units; M0=pre-vaccination; M1=1 month post-dose 1, M2/M6/M13=1, 5 & 12 months post-dose 2. Error bars depict 95% confidence intervals. The first bar in each time period (M) is HZ/su PreChemo, second bar is HZ/su OnChemo, third bar is Placebo PreChemo, and fourth bar is Placebo OnChemo.

FIG. 9 graphs humoral VRR for anti-gE antibody ELISA concentrations (adapted ATP cohort for humoral immunogenicity) in ST subjects, where gE=glycoprotein E; ATP=according-to-protocol; VRR=vaccine response rates; %=percentage of responders; M1=1 month post-dose 1, M2/M6/M13=1, 5 & 12 months post-dose 2. VRR was defined as: (i) for subjects initially seropositive for anti-gE antibodies, post-second vaccination antibody concentration 4-fold above the pre-vaccination level; (ii) for subjects initially seronegative for anti-gE antibodies, post-second vaccination antibody concentration 4-fold above the anti-gE cut-off (97 mIU/mL). Error bars depict 95% confidence intervals. The first bar in each time period (M) is HZ/su PreChemo, second bar is HZ/su OnChemo, third bar is Placebo PreChemo, and fourth bar is Placebo OnChemo.

FIG. 10 graphs frequency of gE-specific CD4²⁺ T-cells (adapted ATP sub-cohort for CMI) in ST subjects, where gE=glycoprotein E; ATP=according-to-protocol; CMI=cell-mediated immunogenicity; M0=pre-vaccination; M1=1 month post-dose 1, M2/M13=1 & 12 months post-dose 2; Min=minimum; Max=maximum; Q1=first quartile; Q3 third quartile.

FIG. 11 graphs CMI VRR by gE-specific CD4²⁺ T-cell frequency (adapted ATP sub-cohort for CMI) in ST subjects, where CMI=cell-mediated immunogenicity; VRR=vaccine response rates; gE=glycoprotein E; ATP=according-to-protocol; %=percentage of responders; M1=1 month post-dose 1, M2=1 month post-dose 2; M13=12 months post-dose 2. Vaccine response rates were defined as: (i) for subjects with initial pre-vaccination anti-gE CD4²⁺ frequencies above the cut-off (320/106 gE-specific CD4²⁺), a ≥2-fold increase in anti-gE CD4²⁺ frequencies as compared to pre-vaccination levels; (ii) for subjects with initial pre-vaccination anti-gE CD4²⁺ frequencies below the cut-off, an anti-gE CD4²⁺ frequency 2-fold above the cut-off. Error bars depict 95% confidence intervals. The first bar in each time period (M) is HZ/su PreChemo, second bar is Placebo PreChemo.

FIG. 12 graphs GMC of anti-gE antibodies (ATP cohort for humoral immunogenicity) in RTR subjects. The first bar at each time point is HZ/su; the second bar is Placebo. GMC=geometric mean concentration; gE=glycoprotein E; ATP=according-to-protocol; IU=international units; M0=pre-vaccination; M1=1 month post-dose 1; M2=1 month post-dose 2; Y=years of age; CIS=calcineurin inhibitor or sirolimus; CS=corticosteroids; MC=mycophenolate compound. Error bars depict 95% confidence intervals.

FIG. 13 graphs humoral VRR for anti-gE antibody ELISA concentrations (ATP cohort for humoral immunogenicity) in RTR subjects. The first bar at each time point is HZ/su; the second bar is Placebo. VRR=vaccine response rates; %=percentage of responders; other abbreviations are the same as in FIG. 12 VRR was defined as: (i) for subjects initially seropositive for anti-gE antibodies, post-second vaccination antibody concentration ≥4-fold above the pre-vaccination level; (ii) for subjects initially seronegative for anti-gE antibodies, post-second vaccination antibody concentration ≥4-fold above the anti-gE cut-off (97 mIU/mL). Error bars depict 95% confidence intervals.

FIG. 14 graphs the frequency of gE-specific CD4²⁺ T cells (ATP sub-cohort for CMI) in RTR subjects. CMI=cell-mediated immunogenicity; Min=minimum; Max=maximum; Q1=first quartile; Q3=third quartile; other abbreviations are the same as in FIGS. 12 and 13.

FIG. 15 graphs CMI VRR by gE-specific CD4²⁺ T cell frequency (ATP sub-cohort for CMI) in RTR subjects. Abbreviations are the same as in FIGS. 12-14. Vaccine response rates were defined as: (i) for subjects with initial pre-vaccination anti-gE CD42+ frequencies above the cut-off (320/106 gE-specific CD42+), a ≥2-fold increase in anti-gE CD42+ frequencies as compared to pre-vaccination levels; (ii) for subjects with initial pre-vaccination anti-gE CD42+ frequencies below the cut-off, an anti-gE CD42+ frequency ≥2-fold above the cut-off. Error bars depict 95% confidence intervals.

### DETAILED DESCRIPTION

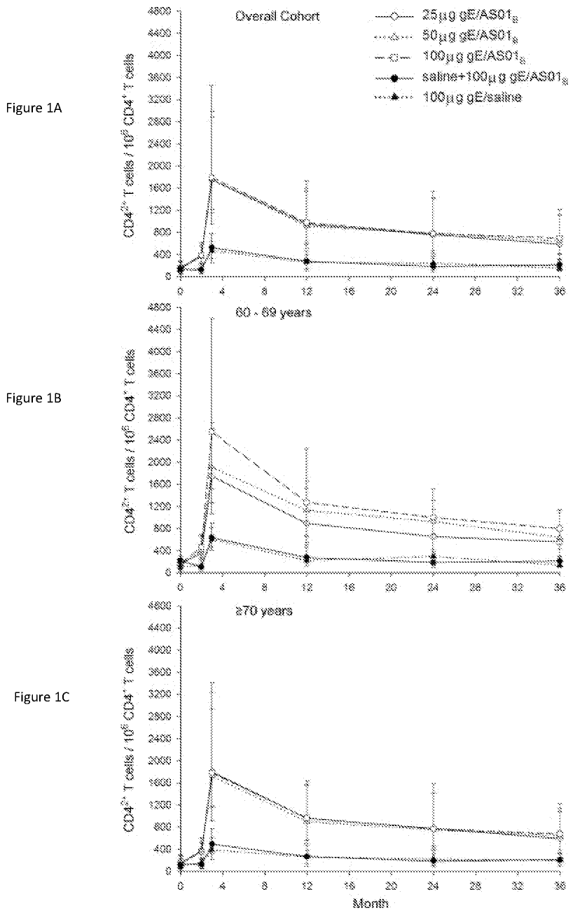

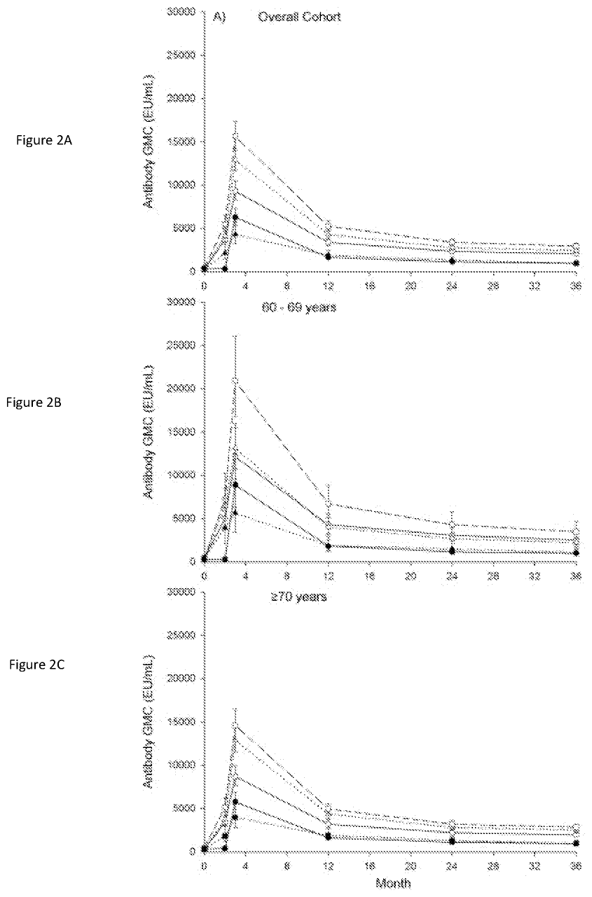

The current invention relates to the unexpected finding of effective protection against, or prevention of or reduction of the severity of shingles and/or PHN following a single or a first dose of the immunogenic composition described herein. Previously, supported by clinical immune data as reported by Chlibek R. et al. (2014 Vaccine 32:1745-1753), the adjuvanted VZV gE subunit vaccine was believed to require at least 2 doses of the vaccine composition in order to generate a sufficient immune response for effectively preventing shingles and/or PHN in a susceptible individual, i.e. in a population known for immune senescence, e.g. older adults (50 years of age (yoa) or older, 60 yoa or older, 70 yoa or older, or 80 yoa or older), or, immunocompromised human individuals (e.g. human individuals undergoing immunosuppressing therapy, individuals suffering immunosuppressing infections such as HIV). FIGS. 1A-C and 2A-C were taken from Chlibek R. et al. (2014 Vaccine 32:1745-1753). In FIGS. 1A-C and 2A-C (as indicated in FIG. 1A), study subjects received either: two doses two months apart of the same adjuvanted (AS01B) VZV gE composition (containing either 25 μg, 50 μg or 100 μg VZV gE); two doses two months apart of unadjuvanted VZV gE (100 μg gE/saline), or one dose of saline followed two months later by one dose of adjuvanted VZV gE (saline+100 μg gE/AS01B). FIGS. 1A-C show a substantial difference in the cell mediated immune response following 0.2 month 2-dose schedule of adjuvanted gE (25 μg, 50 μg or 100 μg gE per dose) compared to a single dose of adjuvanted gE (100 μg gE per dose) in the overall cohort of subjects (FIG. 1A), the cohort of subjects from 60 to 69 years of age (FIG. 1B), and the cohort of subjects greater than or equal to 70 years of age (FIG. 1C). FIG. 2A-C shows a similar effect with respect to the VZV gE antibody levels. Efficacy of adjuvanted VZV gE subunit vaccine compositions following 2-dose vaccination schedule is described in WO2016/096968.

It has now been found that shingles and/or PHN can effectively be prevented or the severity thereof reduced in an individual following one dose of the immunogenic composition described herein. It has been found that one dose is effective in the protection against or prevention of shingles and/or PHN prior to 2 months following the administration of the one dose of the immunogenic, e.g. vaccine, composition described herein. As shown in Example 1, in a population of subjects 50 years of age or older who received a single dose of the immunogenic composition (i.e. subset of study subjects in the study who did not complete the 2-dose schedule), effective protection against HZ was demonstrated in the vaccinated subjects (compared to subjects receiving placebo) during a mean follow-up time of 76 days (see Table 1). In Example 2, data from subjects (70 yoa or older) who received a single dose were pooled with data from subjects (70 yoa or older who had received only the first dose of a two-dose administration schedule). The effective protection against HZ by a single dose of the immunogenic composition was demonstrated in this pooled population (compared to placebo vaccinated subjects) during a mean follow-up time of 85 days (see Table 1).

According to one embodiment, the method comprises administering a single dose of the immunogenic composition, i.e., it is the dose in a single dose immunisation schedule. Alternatively, the method comprises administering one dose that is the first dose administered in a multi-dose immunisation schedule. In a further embodiment, the one dose is the first dose administered in a 2-dose immunisation schedule. In yet a further embodiment, the one dose is the first dose of a 2-dose immunisation schedule wherein the first dose is effective in the prevention of, or protection against, HZ prior to the administration of the second dose of the 2-dose immunisation schedule.

In case of a multi-dose immunisation schedule, the interval in between administration of 2 (or multiple) doses of the vaccine may be varied between 1 month and about one year (i.e. 12 months), or between 1 and 3 months, or between 2 and 12 months, or between 2 and 6 months. In one embodiment the interval is 2, 6 or 12 months. Particularly, the interval is 2 months. Also particularly, the interval is 12 months. Alternatively, the interval is 1 year. It will be apparent to those in the art that a “1 month” interval is not limited to administration of the subsequent dose only on the day occurring exactly one month later; administration on a “1 month” schedule will typically occur during the period from 30 to 48 days after the previous administration. Administration on a 2 months interval will typically be within 49 to 83 days; a 12 month interval will typically be within 335 and 395 days.

In a specific embodiment, the one dose is the first dose administered in a 2-dose immunisation schedule with a 0.2 to 0.6 month interval.

The use or method of preventing or protecting in accordance with the present invention (the vaccination) provides high efficacies following administration of one dose of the immunogenic composition. Efficacy in prevention of or protection against HZ of the one dose of the immunogenic composition is expressed as the reduction of the occurrence of HZ in a population after receiving only one dose of the immunogenic composition, compared to placebo. The efficacy of one dose of the immunogenic composition in the prevention of or protection against HZ is 50% or more, suitably 55% or more, suitably 60% or more, suitably 65% or more, suitably 70% or more, suitably 75% or more, suitably 80% or more, suitably 85% or more, or, 90% or more.

Furthermore, it has been found that the efficacy in accordance with the present invention is high in multiple target populations. Contrary to the usual decrease in vaccine efficacy observed in subjects with a waning immune system, efficacy of vaccination using the immunogenic, e.g. vaccine, composition in accordance with the present invention is exceptionally high in multiple target populations, even in individuals above or older than 70 years of age substantial protection following one dose was achieved.

Particular target populations considered in accordance with the present invention are human individuals ≥50 years of age, ≥60 years of age, ≥70 years of age, between 50 and 59 years of age, or, between 60 and 69 years of age; and more in particular are considered subjects that are ≥70 years of age, such as ≥71 years of age, e.g. ≥72 years of age, such as ≥73 years of age, e.g. ≥74 years of age, such as ≥75 years of age, e.g. ≥80 years of age or ≥81 years of age. In a particular embodiment, the target population comprises human individuals older than 70 years of age.

Accordingly, in specific embodiments:
the efficacy of one dose of the immunogenic composition in the prevention of or protection against HZ is 50% or more, suitably 55% or more, suitably 60% or more, suitably 65% or more in a population of adults of 70 years of age or older,
the efficacy of one dose of the immunogenic composition in the prevention of or protection against HZ is 50% or more, suitably 55% or more, suitably 60% or more, suitably 65% or more, suitably 70% or more, suitably 75% or more, suitably 80% or more, suitably 85% or more, or, 90% or more in a population of adults of 50 years of age or older,
the efficacy of one dose of the immunogenic composition in the prevention of or protection against HZ is 50% or more, suitably 55% or more, suitably 60% or more, suitably 65% or more in a population of adults of 70 years of age or older, as measured within two weeks, one month, six weeks, or within two months, following the one dose;
the efficacy of one dose of the immunogenic composition in the prevention of or protection against HZ is 50% or more, suitably 55% or more, suitably 60% or more, suitably 65% or more, suitably 70% or more, suitably 75% or more, suitably 80% or more, suitably 85% or more, or, 90% or more in a population of adults of 50 years of age or older, as measured within two weeks, one month, six weeks, or within two months, following the one dose.

The dose of the immunogenic composition may be administered in a single dose regimen. As used herein, a ‘single dose’ or a ‘single dose regimen’ means only one dose is administered to achieve prevention or protection. A subject undergoing a single dose regimen is not scheduled or instructed to obtain a second dose, e.g., during the next one year, two years, 18 months, three years, four years, or more. Thus, in a method comprising administration of a single dose of the immunogenic composition, the method comprises an administration step that consists of administering a single dose of the immunogenic composition.

Further particular populations suitable for treatment with the present invention are immune-compromised populations or individuals, such as HIV positive patients or patients suffering from AIDS, transplant patients e.g. renal transplant patients or haematopoietic cell transplant patients, patients suffering haematological malignancies, solid tumor patients or patients otherwise immune deficient or immune compromised.

Additional specific populations of subjects facing declining immunity or immunosuppressing therapy who would benefit from the timely administration of one or a first dose of the immunogenic composition are patients with hematopoietic stem cell transplantation, hematological malignancies, solid organ malignancies, solid organ transplantation, end stage renal diseases, psoriasis, rheumatoid arthritis, systemic lupus erythematosus and inflammatory bowel disease.

Because of the early onset of the effective prevention of HZ following one dose of the immunogenic composition, the protection against or prevention of HZ provided by one dose, e.g. a first dose in a multi-dose schedule, is particularly useful for subjects enrolled to receive immunosuppressant therapy or otherwise faced with a situation where natural immunity will be suppressed in the near future. Accordingly, the invention provides the use of one dose of the immunogenic composition prior to when immunosuppressing therapy is initiated in a subject, or concomitant with the start of immunosuppressing therapy. Thus a particular population suitable for treatment with the present invention are humans who are immune competent (capable of developing an immune response within normal ranges) but who are scheduled to receive, or at increased likelihood of receiving, immunosuppressive therapy in the near future (e.g., within one week, two weeks, three weeks, one month, six weeks, two months, three months, four months, five months, six months, or one year after the administration of the one dose; or between one week to one month, two months, three months, four months, five months, six months or one year after the administration of the one dose; or between two weeks to six weeks, two months, three months, four months, five months, six months, or one year after administration of the one dose; or between one month and six weeks, two months, three months, four months, five months, six months, or one year after administration of the one dose). Suitable subjects include those beginning (or enrolled to receive) immunosuppressant (also referred to as immunosuppressive or immunosuppressing) therapy, such as chemotherapy, radiotherapy, or immunosuppressive pharmaceutical compounds, as well as subjects scheduled to undergo organ transplantation or those enrolled on a waiting list to receive a transplanted organ.

No herpes zoster (HZ) vaccine is currently approved for use in immunosuppressed or immunocompromised individuals. The HZ incidence in individuals with solid tumors (ST) receiving immunosuppressive chemotherapy (chemo) is estimated as 3-4 times higher than in the overall US population (12/1000 vs 3.2/1000 person-years) (Habel et al., Cancer Epidemiol Biomarkers Prev, 2013; 22:82-90; Insinga et al., J Gen Intern Med 2005; 20:748-53). The incidence rate of herpes zoster in individuals with solid organ transplants (SOTs) is estimated as 8-9 times higher than the rate in the overall US population (3.2/1000 person-years).

In a further embodiment, the target population considered in accordance with the present invention comprises or consists of subjects previously vaccinated with a live attenuated VZV vaccine. HZ and/or PHN protection following immunisation using live-attenuated VZV vaccine has been reported to wane rapidly (Tseng H F et al. J Infect Dis (2016) 213(12):1872-5). It has now been found that the immunogenic composition described herein can effectively be used in the prevention of shingles (or HZ) and/or PHN in subjects who were previously vaccinated using live-attenuated VZV vaccine, such as more than 3 years earlier, 4 years earlier, more than 5 years earlier, more than 6 years earlier, more than 7 years earlier, more than 8 years earlier, or more than 10 years earlier.

The immunogenic, e.g. vaccine, composition in accordance of the invention comprises a recombinant VZV gE antigen in combination with an adjuvant.

As disclosed herein, a suitable VZV gE antigen is the VZV glycoprotein gE (also known as gp1) or an immunogenic variant thereof, truncated to remove the carboxy terminal anchor region. The complete varicella-zoster virus (VZV) nucleotide sequence was disclosed by Davison et al. (J Gen Virol, 67:1759-1816 (1986)). The wild type or full length gE protein consists of 623 amino acids comprising a signal peptide, the main part of the protein, a hydrophobic anchor region (residues 546-558) and a C-terminal tail. In one aspect, a VZV gE C-terminal truncate (also referred to truncated gE or gE truncate) is used whereby the truncation removes 4 to 20 percent of the total amino acid residues from the carboxy terminal end, e.g. lacking residues 547 to 623. In an alternative embodiment, the truncated gE lacks the carboxy terminal anchor region (e.g. by an internal deletion in the C-terminal region, suitably approximately amino acids 547-558 of the wild type sequence). In one embodiment, VZV gE antigen is a truncated gE comprising or consisting of the sequence of SEQ ID NO. 1. In a further embodiment, the VZV gE antigen is not presented in the form of a fusion protein comprising a further (non-gE) VZV protein or immunologically active fragment thereof.

The VZV gE antigen, including anchorless VZV gE antigens (which are also immunogenic variants) and production thereof are described in EP0405867 (incorporated herein by reference) and references therein [see also Vafai A. Antibody binding sites on truncated forms of varice Ila-zoster virus gpl(gE) glycoprotein Vaccine 1994 12:1265-9]. EP0192902 also discloses gE and production thereof. Truncated gE is also disclosed by Haumont et al. Virus Research (1996) vol 40, p 199-204, herein incorporated fully by reference. An adjuvanted VZV gE composition suitable for use in accordance with the present invention is disclosed in WO2006/094756 (U.S. Pat. No. 7,939,084, which is incorporated herein by reference), i.e. a carboxy terminally truncated VZV gE in combination with an adjuvant comprising QS21, 3D-MPL and liposomes further containing cholesterol. Leroux-Roels I. et al. (J. Infect. Diseases 2012:206 1280-1290) reported on a phase I/II clinical trial evaluating the adjuvanted VZV truncated gE subunit vaccine.

As used herein the term “variant” refers to an antigen that is modified relative to its naturally occurring form. As disclosed herein, a suitable “variant” is an “immunogenic variant”, thus which is sufficiently similar to native antigens to retain antigenic properties and remains capable of inducing an immune response which is cross-reactive with the native antigen. A variant polypeptide may contain a number of substitutions, preferably conservative substitutions, i.e. a substitution of one amino acid by another one with similar properties such as the aliphatic amino acids Val, lie, Leu, Met or basic amino acids Lys, Arg, His or aromatic amino acids Phe, Tyr, Trp, (for example, 1-50, such as 1-25, in particular 1-10, or 1, 2, 3, 4, 5, 6, 7, 8, 9 or 10 alterations, and especially 1 amino acid residue(s) may be altered, e.g. substituted or deleted) when compared to the reference sequence, i.e. wild type sequence. In particular, variants with respect to SEQ ID No. 1 are contemplated. Suitably such substitutions do not occur in the region of a major epitope (e.g. immunologically important epitope), and do not therefore have a significant impact on the immunogenic properties of the antigen. VZV gE is known to contain B cell and CD4+ T cell epitopes as disclosed by R. E. Bergen et al. (Viral Immunology, 4 (3) (1991), pp. 151-166), W. J. Fowler et al. (Virology, 214 (2) (1995), pp. 531-540), G. N. Malavige et al. (Clin Exp Immunol, 152 (3) (2008), pp. 522-531) and L. Wu & B. Forghani (Arch Virol, 142 (2) (1997), pp. 349-362). Protein variants may also include those wherein additional amino acids are inserted compared to the reference sequence, for example, such insertions may occur at 1-10 locations (such as 1-5 locations, suitably 1 or 2 locations, in particular 1 location) and may, for example, involve the addition of 50 or fewer amino acids at each location (such as 20 or fewer, in particular 10 or fewer, especially 5 or fewer). Suitably such insertions do not occur in the region of an epitope, and do not therefore have a significant impact on the immunogenic properties of the antigen. One example of insertions includes a short stretch of histidine residues (e.g. 2-6 residues) to aid expression and/or purification of the antigen in question. Variants also include those wherein amino acids have been deleted compared to the reference sequence, for example, such deletions may occur at 1-10 locations (such as 1-5 locations, suitably 1 or 2 locations, in particular 1 location) and may, for example, involve the deletion of 20 or fewer amino acids at each location (such as 10 or fewer, in particular 5 or fewer, especially 2 or fewer). Suitably such deletions do not occur in the region of an epitope, and do not therefore have a significant impact on the immunogenic properties of the antigen. The skilled person will recognise that a particular protein variant may comprise substitutions, deletions and additions (or any combination thereof). Variants preferably exhibit at least about 70% identity, more preferably at least about 80% identity and most preferably at least about 90% identity (such as at least about 95%, at least about 98% or at least about 99%) to the associated reference sequence. Examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Whether or not a given variant raises such an immune response, may be measured by a suitable immunological assay such as an ELISA or flow cytometry.

The amount of VZV gE antigen used in the immunisation of human individuals against HZ or PHN is selected as an amount which induces an immunoprotective response without significant, adverse side effects in typical vaccinees. Such amount will vary depending upon which specific antigen is employed and how it is presented. Generally, it is expected that each dose will comprise 1-1000 μg of protein, such as 2-100 μg, or 5-60 μg. Where VZV gE antigen is used then in one aspect 25-100 μg of gE may be used in humans, such as 40-100 μg of gE for human use, in one aspect about 25 μg, about 50 μg or about 100 μg of μg, suitably 25 μg, 50 μg or 100 μg of gE. In a preferred embodiment, VZV gE antigen (e.g. of SEQ ID NO. 1) is used in a 50 μg dose. As disclosed herein, “dose” is the amount administered in a single administration.

As disclosed herein, a suitable adjuvant comprises a TLR-4 ligand, and a saponin in a liposomal formulation.

A particularly suitable saponin for use in the present invention is Quil A and its derivatives. Quil A is a saponin preparation isolated from the South American tree Quillaja saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fractions of Quil A have been isolated by HPLC which retain adjuvant activity without the toxicity associated with Quil A (EP 0 362 278), for example QS7 and QS21 (also known as QA7 and QA21). QS21 is a natural saponin derived from the bark of Quillaja saponaria Molina, which typically induces CD8+ cytotoxic T cells (CTLs), Th1 cells and a predominant IgG2a antibody response and is a preferred saponin in the context of the present invention.

Suitably, the saponin is provided in its less reactogenic composition where it is quenched with an exogenous sterol. Suitable sterols include β-sitosterol, stigmasterol, ergosterol, ergocalciferol and cholesterol. In one particular embodiment, the adjuvant composition comprises cholesterol as sterol. These sterols are well known in the art, for example cholesterol is disclosed in the Merck Index, 11th Edn., page 341, as a naturally occurring sterol found in animal fat. Several particular forms of less reactogenic compositions wherein QS21 is quenched with an exogenous cholesterol exist. The saponin/sterol is formulated in a liposomal formulation structure. Methods for obtaining saponin/sterol in a liposomal formulation are described in WO 96/33739 (U.S. Pat. No. 6,846,489, incorporated herein by reference), in particular Example 1. The relative amount of sterol to phospholipid is 1-50% (mol/mol), suitably 20-25%.

Where the active saponin fraction is QS21, the ratio of QS21:sterol will typically be in the order of 1:100 to 1:1 (w/w), suitably between 1:10 to 1:1 (w/w), and preferably 1:5 to 1:1 (w/w). Suitably excess sterol is present, the ratio of QS21:sterol being at least 1:2 (w/w). In one embodiment, the ratio of QS21:sterol is 1:5 (w/w). The sterol is suitably cholesterol.

The adjuvant composition comprises a TLR-4 agonist. A suitable example of a TLR-4 agonist is a lipopolysaccharide, suitably a non-toxic derivative of lipid A, particularly monophosphoryl lipid A or more particularly 3-Deacylated monophoshoryl lipid A (3D-MPL).

3D-MPL is sold under the name MPL by GlaxoSmithKline Biologicals S.A. and is referred throughout the document as MPL or 3D-MPL. See, for example, U.S. Pat. Nos. 4,436,727; 4,877,611; 4,866,034 and 4,912,094 (each of which incorporated herein by reference). 3D-MPL primarily promotes CD4+ T cell responses with an IFN-g (Th1) phenotype. 3D-MPL can be produced according to the methods disclosed in GB 2 220 211 A. Chemically it is a mixture of 3-deacylated monophosphoryl lipid A with 4, 5 or 6 acylated chains. In the compositions of the present invention small particle 3D-MPL may be used to prepare the adjuvant composition. Small particle 3D-MPL has a particle size such that it may be sterile-filtered through a 0.22 μm filter. Such preparations are described in WO 94/21292. Preferably, powdered 3D-MPL is used to prepare the adjuvant compositions of the present invention.

Other TLR-4 agonists which can be used are alkyl Glucosaminide phosphates (AGPs) such as those disclosed in WO98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR-4 agonists, and some are TLR-4 antagonists. In the present invention, the use of a TLR-4 agonist is contemplated.

Other suitable TLR-4 ligands are as described in WO2003/011223 (US20020176861) and in WO 2003/099195 (U.S. Pat. No. 7,833,993), both incorporated herein by reference, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057m ER804058, ER804059, ER804442, ER804680 and ER804764. For example, one suitable TLR-4 ligand is ER804057.

Other TLR4 agonists which may be of use in the present invention include Glucopyranosyl Lipid Adjuvant (GLA) such as described in WO2008/153541 or WO2009/143457 or the literature articles Coler R N et al. (2011) Development and Characterization of Synthetic Glucopyranosyl Lipid Adjuvant System as a Vaccine Adjuvant. PLoS ONE 6(1): e16333. doi:10.1371/journal.pone.0016333 and Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144. WO2008/153541 or WO2009/143457 are incorporated herein by reference for the purpose of defining TLR4 agonists which may be of use in the present invention.

The adjuvant composition comprises both saponin and a TLR4 agonist. In a specific example, the adjuvant composition comprises QS21 and 3D-MPL.

A TLR-4 agonist such as a lipopolysaccharide, such as 3D-MPL, can be used at amounts of between 1 and 100 μg per human dose of the adjuvant composition. 3D-MPL may be used at a level of about 50 μg, for example between 40-60 μg, suitably between 45-55 μg or between 49 and 51 μg or 50 μg. In a further embodiment, the human dose of the adjuvant composition comprises 3D-MPL at a level of about 25 μg, for example between 20-30 μg, suitable between 21-29 μg or between 22-28 μg or between 23 and 27 μg or between 24 and 26 μg, or 25 μg.

A saponin, such as QS21, can be used at amounts between 1 and 100 μg per human dose of the adjuvant composition. QS21 may be used at a level of about 50 μg, for example between 40-60 μg, suitably between 45-55 μg or between 49 and 51 μg or 50 μg. In a further embodiment, the human dose of the adjuvant composition comprises QS21 at a level of about 25 μg, for example between 20-30 μg, suitable between 21-29 μg or between 22-28 μg or between 23 and 27 μg or between 24 and 26 μg, or 25 μg. QS21 may be present at a dose 60 μg, 55 μg or 30 μg per dose. QS21 may be present in a dose ≥20 μg, 40 μg or 45 μg per dose.

The weight ratio of TLR-4 agonist to saponin is suitably between 1:5 and 5:1, suitably 1:1. For example, where 3D-MPL is present at an amount of 50 μg or 25 μg, then suitably QS21 may also be present at an amount of 50 μg or 25 μg per human dose of the adjuvant composition.

By “liposomal formulation” is meant that the saponin and TLR-4 agonist are formulated with liposomes. The liposomes intended for the present invention contain a neutral lipid, for example phosphatidylcholine, which is suitably non-crystalline at room temperature, for example eggyolk phosphatidylcholine, dioleoyl phosphatidylcholine (DOPC) or dilauryl phosphatidylcholine. In a preferred embodiment, the liposomes of the present invention contain DOPC. The liposomes may also contain a charged lipid which increases the stability of the lipsome-QS21 structure for liposomes composed of saturated lipids. In these cases the amount of charged lipid is suitably 1-20% w/w, preferably 5-10%.

WO2013/041572 (US20140234403, incorporated herein by reference), in particular examples 3 and 4, further discloses methods for making a liposome bulk preparation of DOPC liposomes further containing cholesterol and 3D-MPL, for further mixing with QS21, thereby obtaining an adjuvant suitable for use in accordance with the present invention.

In a specific embodiment the immunogenic composition for use according the present invention consists essentially of a VZV gE antigen truncated to remove the carboxy terminal anchor region or derivative thereof, in combination with an adjuvant comprising a QS21, a 3D-MPL and liposomes comprising cholesterol.

The composition is typically administered via the intramuscular route, although alternative routes may be considered, e.g. intradermal or subcutaneous.

The immunogenic, e.g. vaccine, composition in accordance with the invention is for use in vaccination, namely the protection against or prevention of herpes zoster (HZ), i.e. prevention of reactivation of VZV, also referred to as shingles, and/or post herpetic neuralgia (PHN) of a human individual. In one embodiment, the immunogenic, e.g. vaccine, composition is used in the protection against or the prevention of the incidence of herpes zoster. Where HZ does occur then the severity of shingles is suitably reduced compared to an unvaccinated individual (i.e. amelioration of HZ). Also, when HZ does occur, other disease syndromes may develop such as post herpetic neuralgia.

PHN is the most common severe complication of HZ. PHN is defined as pain that persists after the resolution of the HZ rash. Affected patients typically report burning, throbbing, intermittent sharp or electric shock-like pain, or allodynia. Older age is a clear risk factor for PHN. Other risk factors may include a severe HZ rash and a painful HZ prodrome. PHN tends to improve over a period of months. About 70-80% of cases resolve within 1 year, however, in some persons PHN persists for many years (Dworkin et al. 2007. Clin. Infec. Dis.; 44 Suppl. 1: S1-S26). PHN is commonly defined as pain 90 days after rash onset. The intensity, character and duration of PHN vary widely among individuals. Accordingly, a specific questionnaire aimed at evaluating the pain (in terms of magnitude and duration) and discomfort associated with HZ has been specifically designed, called the Zoster Brief Pain Inventory (ZBPI). A copy of said ZBPI questionnaire is available for example in Coplan et al. 2004. J. Pain. 5(6):344-356. This ZBPI is particularly useful, and is routinely used, when assessing, for example in clinical trials, compounds aimed at preventing or protecting against HZ-associated pain, including PHN.

In a further embodiment, the invention relates to the use in the protection against or the prevention of post herpetic neuralgia. Where PHN does occur then the severity of the PHN is suitably reduced compared to an unvaccinated individual (i.e. amelioration of PHN). The use or method as disclosed herein will boost an immune response typically induced by natural infection. As disclosed herein, it is understood that “prevention of” or “protection against” HZ and/or PHN occurs when the incidence and/or severity of the occurrence of HZ and/or PHN is reduced. Prevention of or protection against HZ and/or PHN may be assessed in an identified population as compared to another population, e.g., in a vaccinated population compared to a comparable but unvaccinated population. Reduction of severity means reduction of overall disease, or any of the clinical manifestations associated with HZ and/or PHN. For example, reduction of severity means a reduction of the pain associated with HZ and/or PHN, which pain can be suitably measured and monitored using the ZBPI questionnaire.

In further embodiments, the use or method according to the invention is to protect against or prevent both HZ and PHN.

Even more preferred are each of the foregoing preferred and particularly preferred embodiments wherein the VZV gE antigen has the sequence of SEQ ID No. 1 and is present in a dose of 50 μg, and wherein QS21 and 3D-MPL are also present in a dose of 50 μg.

A further particular embodiment is an immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals 80 years of age or older reducing the incidence of PHN by at least 60% or at least 70%, for at least 5 years.

### Terms

As used herein, a truncated antigen or protein is one that is lacking a region of amino acids, compared to its wild type or full-length version. A “truncated” antigen or protein may be the result of removing the region from a wild type or full-length molecule, or may be made de novo, e.g., produced recombinantly in the truncated form.

It will be understood that “prevention of disease” does not imply prevention of disease in 100% of subjects treated.

As used herein, “immnosuppressant medical therapy” includes treatment with immunosuppressant pharmaceutical compounds. Immunosuppressant pharmaceutical compounds are drugs that suppress or reduce the strength of the body's immune system. When used to lessen the risk of rejection of a transplanted organ, immunosuppressant medical therapy may be referred to as anti-rejection therapy or as the use of anti-rejection pharmaceutical compounds. Additionally, immunosuppressant medical therapy may be used to treat auto-immune disorders such as lupus, psoriasis, and rheumatoid arthritis. Immunosuppressant pharmaceutical compounds include corticosteroids such as prednisone, budesonide, and prednisolone; calcineurin inhibitors such as cyclosporines, tacrolimus; mTor inhibitors such as sirolimus, everolimus; IMDH (inosine monophosphate dehydrogenase) inhibitors such as azathioprine, lefunomide, mycophenolate; and biologics such as monoclonal antibodies The present invention is illustrated by the following, non-limiting examples.

### Example 1-Vaccine Efficacy Against HZ in Adults Aged 50 Years and Older

Example 1 describes the results of a phase III, randomized, observer-blind, placebo-controlled, multicentre, clinical vaccination trial (Trial I) demonstrating the prophylactic efficacy, safety, and immunogenicity of a candidate HZ subunit vaccine, i.e. GSK Biologicals' VZV gE/AS01B vaccine (HZ/su), when administered intramuscularly on a 0, 2-month schedule in adults aged 50 years and older.

The study population includes males and females without severely immune-compromising conditions in the age ranges 50-59 years of age (YOA), 60-69 YOA, 70-79 YOA and 80 YOA. The 70-79 YOA and ≥80 YOA strata were combined for primary analyses. Apportionment of approximately 20-25% of the ≥70 YOA cohort to persons ≥80 YOA ensured that this particularly vulnerable population is adequately represented.

The candidate HZ vaccine tested in this trial is an adjuvanted recombinant VZV gE vaccine as described herein. A saline solution is included as a negative control (placebo) in this study to evaluate the efficacy and safety profile of the candidate HZ vaccine.

The objectives of the clinical vaccination trial included evaluation of vaccine efficacy in the prevention of HZ compared to placebo in subjects within each of the following age ranges: 50-59 YOA, 60-69 YOA and 70 YOA, as measured by the reduction in HZ risk.

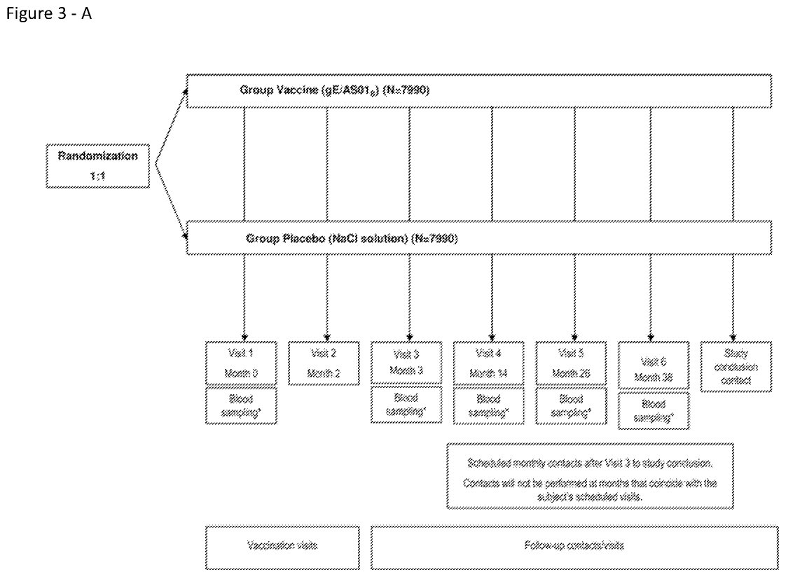

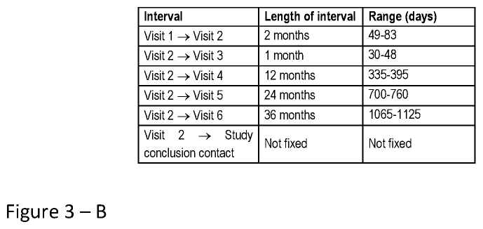

The study design is illustrated by FIGS. 3A and 3B.

The study encompassed two treatment groups, a placebo group and a vaccine group. The placebo group received NaCl solution as a control. The NaCl solution was provided in monodose vials (0.5 mL/dose) containing 150 mM NaCl per 0.5 mL dose. The vaccine group received the study vaccine. Each 0.5 mL dose of study vaccine contained 50 μg of VZV gE antigen, 50 μg of 3D-MPL, 50 μg of QS21, and liposomes (DOPC+ cholesterol). The study vaccine was supplied in 2 vials, one containing the VZV gE antigen, and the other containing Adjuvant System AS01B.
The AS01B Adjuvant System is provided as a liquid formulation in monodose vials, each vial containing at least 0.5 mL of adjuvant. One 0.5 mL dose of AS01B formulation contains 50 μg of 3D-MPL and 50 μg of QS21 mixed with liposomes. The adjuvant system was formulated according to the method of preparation disclosed in example 3 and 4 of WO 2013/041572.
The VZV gE antigen was truncated gE having the sequence of SEQ ID NO: 1. The antigen was obtained according to the method described in Example 2 of WO2006/094756, incorporated herein by reference in its entirety. The VZV gE antigen was provided in a lyophilized form in monodose vials.

Each vial contained 62.5 μg of recombinant purified gE and formulation excipients. Therefore, when the 62.5 μg of VZV gE in each vial was reconstituted with the full volume of the AS01B adjuvant, each vaccine dose contained 50 μg of the VZV gE antigen per 0.5 mL dose of reconstituted vaccine.

The vaccination schedule was two doses of study vaccine or control saline for vaccine group and placebo group respectively, with the first dose at month 0 (visit 1) and second dose at month 2 (visit 2). The vaccine was administered intramuscularly.

Eligible subjects were randomized to vaccine/placebo group according to a 1:1 ratio (vaccine:placebo). Subjects were stratified by age: 50-59 YOA; 60-69 YOA; 70-79 YOA and 80 YOA in approximately an 8:5:3:1 ratio. The 70-79 YOA and ≥80 YOA strata were combined for primary analyses.

Primary HZ efficacy analysis occurred when the following condition was met: at least 196 confirmed HZ cases are accrued in the modified Total Vaccinated cohort (mTVc). The Total Vaccinated cohort (TVc) includes all vaccinated subjects with respect to the vaccine actually administered. The mTVc is the primary cohort for analysis of efficacy which excludes subjects in the TVc for efficacy analysis who were not administered with the second vaccination or who develop a confirmed case of HZ prior to 1 month after the second vaccination. Vaccine efficacy following the 2-dose immunisation schedule for which the tested composition was designed is reported by Himal L. et al. 2015 NEJM 372(22):2087.

Reported herein is the unexpected finding of HZ efficacy of the investigational vaccine composition following a single dose only. Analysis of the data obtained from subjects who enrolled in the study but did not complete the prescribed 2-dose regimen showed, in the overall population of adults 50 yoa and older, effective protection against HZ upon administration of the single dose (see table 1). The mean follow up time is 76 days.

TABLE 1
outcome of the efficacy analysis following one dose of the vaccine composition

| | Vaccine group | | | Placebo group | | | Vaccine Efficacy | | | |
|---|---|---|---|---|---|---|---|---|---|---|
| | | | n/T | | | n/T | | 95% CI | | |
| Age strata | N | n | (per 1000) | N | n | (per 1000) | (%) | LL | UL | p-value |
| ≥50 | 7695 | 2 | 1.1 | 7710 | 20 | 12.5 | 91.27 | 64.01 | 99.01 | <0.0001 |

N = number of subjects included in each group
n = number of subjects having at least one herpes zoster (HZ) confirmed case
n/T (per 1000)= Incidence rate of subjects reporting at least one event per year
LL, UL = 95% Lower and Upper confidence limits
CI = Confidence Interval

### Example 2—Vaccine Efficacy Against HZ in Adults Aged 70 Years and Older

Concurrent to the phase III trial as described in example 1, a further phase III trial (Trial II) was conducted in adults aged 70 years of age and older. The further phase III trial is a randomized, observer-blind, placebo-controlled, multicentre, clinical vaccination trial assessing the prophylactic efficacy, safety, and immunogenicity of a candidate HZ vaccine, i.e. GSK Biologicals' VZV gE/AS01B vaccine, when administered intramuscularly on a 0, 2-month schedule in adults aged 70 years and older.

The study population included males and females without severely immune-compromising conditions in the age ranges of 70-79 years of age (YOA) and 80 YOA. Apportionment of approximately 20-25% of the 70 YOA cohort to persons 80 YOA ensured that this particularly vulnerable population was adequately represented.

The candidate HZ vaccine tested in this trial was an adjuvanted recombinant VZV gE vaccine as described herein. A saline solution was included as a negative control (placebo) in this study to evaluate the efficacy and safety profile of the candidate HZ vaccine.

The objectives of the clinical vaccination trial included evaluation of vaccine efficacy in the prevention of HZ compared to placebo in subjects 70 YOA, as measured by the reduction in HZ risk.

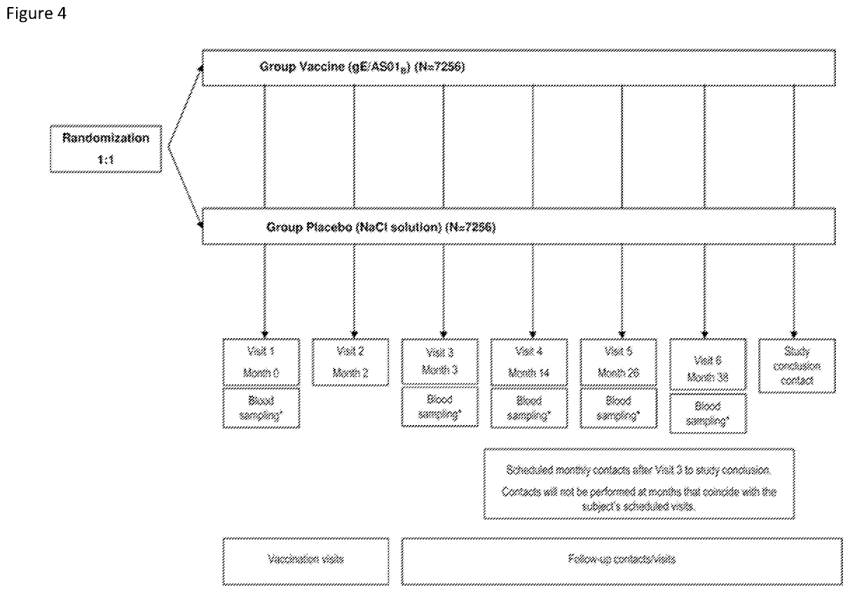

The study design is illustrated by FIG. 4.

The study encompassed two treatment groups, a placebo group and a vaccine group. The placebo group received NaCl solution as a control. The NaCl solution was provided in monodose vials (0.5 mL/dose) containing 150 mM NaCl per 0.5 mL dose. The vaccine group received the study vaccine. Each 0.5 mL dose of study vaccine contained 50 μg of VZV gE antigen, 50 μg of 3D-MPL, 50 μg of QS21, and liposomes (DOPC+ cholesterol). The study vaccine was supplied in 2 vials, one containing the VZV gE antigen, and the other containing Adjuvant System AS01B. The AS01B adjuvant and the VZV gE antigen are as described in Example 1.

Eligible subjects were randomized to vaccine/placebo group according to a 1:1 ratio (vaccine:placebo). Subjects were stratified by age: 70-79 YOA and ≥80 YOA in approximately a 3:1 ratio. The 70-79 YOA and ≥80 YOA strata were combined for primary analyses.

Based on the efficacy results obtained in the trial described in Example 1, the statistical power of vaccine efficacy in the prevention of HZ in the present trial has been re-evaluated, and as a result, primary HZ efficacy analysis was re-evaluated to occur when the following condition was met: at least 211 confirmed HZ cases were accrued in the modified Total Vaccinated cohort (mTVc). The Total Vaccinated cohort (TVc) includes all vaccinated subjects with respect to the vaccine actually administered. The mTVc is the primary cohort for analysis of efficacy which excludes subjects who were not administered with the second dose of vaccine or placebo or who developed a confirmed case of HZ prior to a month after the second dose.

Because of the limited data available after one dose in Trial II, the data with regard to the subjects 70 yoa or older from Trial I were pooled with the Trial II data in order to assess efficacy of the vaccine following one dose of the vaccine in individuals 70 yoa or older. The resulting analysis is provided in table 2. The mean follow up time was 85 days.

TABLE 2

| | Vaccine group | | | Placebo group | | | Vaccine Efficacy | | | |
|---|---|---|---|---|---|---|---|---|---|---|
| | | | n/T | | | n/T | | 95% CI | | |
| Age strata | N | n | (per 1000) | N | n | (per 1000) | (%) | LL | UL | p-value |
| Overall ≥70 | 8758 | 7 | 3.2 | 8773 | 20 | 10.5 | 69.51 | 24.94 | 89.11 | <0.0001 |

N = number of subjects included in each group
n = number of subjects having at least one herpes zoster (HZ) confirmed case
n/T (per 1000) = Incidence rate of subjects reporting at least one event per year
LL, UL = 95% Lower and Upper confidence limits
CI, Confidence Interval

### Example 3—Vaccine Efficacy Against HZ in Adults Previously Vaccinated with a Live-Attenuated Herpes Zoster Vaccine

The present example reports on a phase III, open-label, group-matched, multi-centre study. Adults ≥65 years of age who were previously vaccinated with a live-attenuated VZV vaccine, also referred to as ZVL, (ZOSTAVAX) ≥5 years prior to study start (Group 1: HZ-PreVac) and group matched ZVL-naïve adults (Group 2: HZ-NonVac) were enrolled.

Participants in the HZ-NonVac-group were group-matched to those in the HZ-PreVac-group according to the predefined variables age (65-69, 70-79, ≥80), gender, race (Caucasian, African American, Hispanic and Other) and medical condition. Medical conditions were ranked in a hierarchical order (immune-mediated diseases, diabetes mellitus, current depression, pulmonary disorders, heart conditions, none of these medical conditions), and subjects were matched according to the highest ranked condition.

Study participants were men or women aged 65 years or older at the time of the first vaccination with VZV gE/AS01B. Adults eligible for inclusion in the HZ-PreVac-group had received ZVL at least 5 years prior to study start. Adults were excluded from participation if they had received or were scheduled to receive a live vaccine within 30 days, had received any investigational or non-registered drug or vaccine within 30 days, had received immuno-suppressants or other immune-modifying drugs for more than 14 consecutive days within 180 days, or had received any long-acting immune-modifying drugs within 180 days before the first VZV gE/AS01B vaccination. Adults with a history of HZ, or adults scheduled to receive a HZ vaccine other than VZV gE/AS01B, as well as adults with a history of any reaction or hypersensitivity to any of the vaccine components, were excluded from participation.

Study objectives. The co-primary objectives of the study were to compare the humoral immune responses 1 month after dose 2 of VZV gE/AS01B between the HZ-PreVac- and HZ-NonVac-groups, and to evaluate safety and reactogenicity up to 1 month after dose 2 of VZV gE/AS01 in both study groups. The secondary study objectives also presented in this manuscript were to assess the humoral and CMI responses to the VZV gE/AS01B vaccine at baseline (pre-vaccination), and 1 month post-dose 1 and post-dose 2 in both study groups.

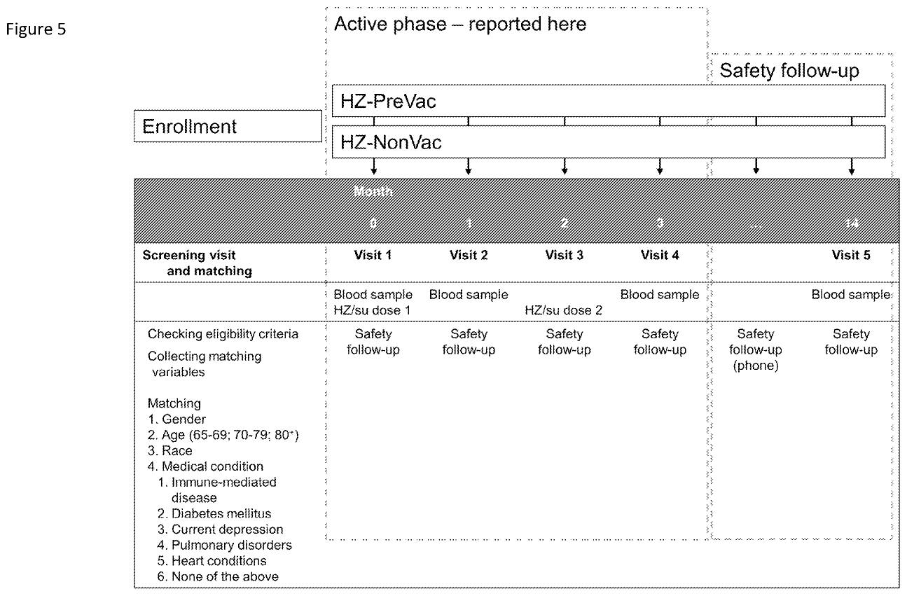

Assessment ofimmunogenicity. Blood samples for the immunogenicity assessments were collected at baseline, and 1 month after the first and second vaccine doses (FIG. 5). Anti-gE antibody concentrations were measured by anti-gE ELISA. The assay cut-off was 97 mIU (International Units)/mL. CMI responses were assessed by intracellular cytokine staining and flow cytometry. Briefly, peripheral blood mononuclear cells were stimulated in vitro with gE peptides, after which frequencies of gE-specific CD4+ T cells expressing at least 2 activation markers (here referred to as CD4²⁺) of the 4 markers assessed (interferon-γ, interleukin-2, tumor necrosis factor-α and CD40 ligand) were determined.

Statistical Analyses. All statistical analyses were performed using the Statistical Analysis Systems (SAS) version 9.3 TS1M2 on windows SDD 4.3.3.

Immunogenicity data were analysed on the according-to-protocol cohort, which included all participants who complied with protocol-specified procedures and for whom data were available. For inferential analyses of the co-primary endpoint data, an Analysis of variance (ANOVA) model was used on log-transformed antibody concentration data and included the vaccine group and the group-matching categories as fixed effects. Adjusted means and a difference of means between both study groups were calculated together with 2-sided CIs and back-transformed to the original units to provide adjusted geometric mean concentrations (GMCs) and GM ratios. Per protocol, non-inferiority of the response was demonstrated if the upper limit of the 2-sided CI of the adjusted GMC ratio of the HZ-NonVac over the HZ-PreVac group at 1 month post-dose 2 (active phase) was below 1.5. Secondary immunogenicity endpoint data, including CMI data presented here, were evaluated using descriptive analyses. For descriptive data, the 95% CI for GMCs was obtained for each group separately. First, a 95% CI for the mean of log-transformed concentrations was obtained, under the assumption that log-transformed values were normally distributed with unknown variance.

Subsequently, the 95% CI for GMCs was calculated by anti-log transformation the previously calculated 95% CI for the mean of log-transformed concentrations.

Based on variability in the anti-gE antibody response to VZV gE/AS01B as seen in previous clinical trials, a sample size of 190 evaluable participants per study group would demonstrate non-inferiority in humoral immunogenicity with at least 99% power.

Results

Participants. A total of 822 older adults were screened for participation in this study. Of these, 215 people not previously vaccinated were matched according to pre-specified criteria (age, gender, geographic ancestry and medical condition) to 215 people who had previously been vaccinated with ZVL. (FIG. 2). Of the 430 vaccinated participants, 425 (98.8%) completed the active phase of the study. Demographic characteristics were comparable for participants in both study groups and are presented in Table 3.

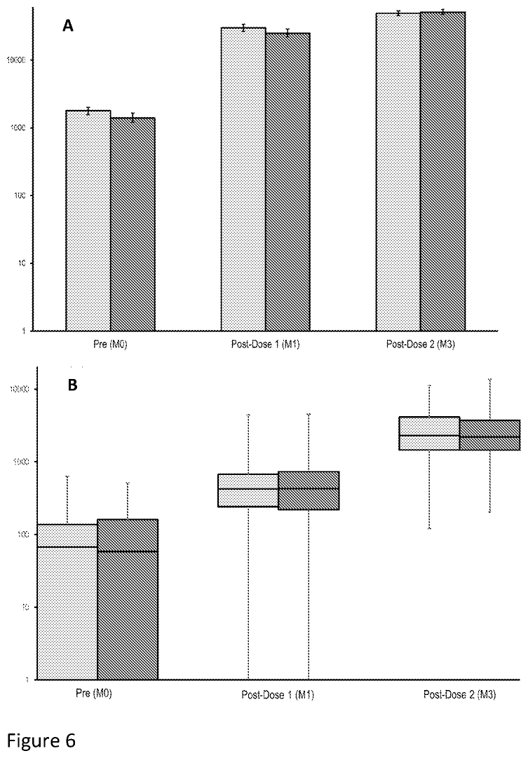

Immunogenicity. Prior to the first vaccination, all evaluable participants in the HZ-PreVac group and 98% of evaluable participants in the HZ-NonVac group were seropositive for anti-gE antibodies (anti-gE concentration above the assay cut-off of 97 mIU/mL). Anti-gE antibody GMCs appeared similar at baseline in both study groups and increased markedly after both vaccine doses (FIG. 6A and Table 4). Anti-gE antibody GMCs post-dose 2 were comparable for both study groups, with an adjusted GMC ratio of 1.04 (Table 5). The primary immunologic study objective was met, as the upper limit of the adjusted GMC ratio of the HZ-NonVac group over the HZ-PreVac group was below the 1.5 cut-off (Table 5).

At baseline, the median CD4²⁺ T cell frequency appeared similar in both groups. After dose 1, median frequencies of gE-specific CD4²⁺ T cells increased in both groups, and a more substantial overall increase was seen after dose 2 (FIG. 6B and Table 4). No difference in CD4²⁺ T cell frequency was apparent between study groups.

### CONCLUSION

This study showed that the humoral immune response to VZV gE/AS01B 1 month post-dose 2 was non-inferior in adults over 65 years of age who were vaccinated with the live-attenuated zoster vaccine (ZOSTAVAX) over 5 years ago when compared to adults who never received this vaccination. This study showed that prior vaccination with ZVL does not negatively impact the humoral immune responses to VZV gE/AS01B. In addition, descriptive analyses did not reveal any apparent differences in CMI responses as assessed by CD4²⁺ T-cell frequencies and post-vaccination increases in CD4²⁺ T cell-frequencies were observed in both study groups.

TABLE 3
Characteristics of study participants (Total vaccinated cohort)

| | | Total | | HZ-NonVac | | HZ-PreVac | |
|---|---|---|---|---|---|---|---|
| Characteristic | Parameters/Categories | N = 430 | | N = 215 | | N = 215 | |
| Age | Mean (±SD) | 70.9 | (4.6) | 70.8 | (4.6) | 71.1 | (4.5) |
| Gender | Female n (%) | 220 | (51.2) | 111 | (51.6) | 109 | (50.7) |
| | Male n (%) | 210 | (48.8) | 104 | (48.4) | 106 | (49.3) |
| Geographic | Caucasian/European n (%) | 430 | (100) | 215 | (100) | 215 | (100) |
| ancestry | | | | | | | |

HZ-NonVac = participants who never received the live-attenuated zoster vaccine (ZVL); HZ-PreVac = participants who received ZVL ≥5 years prior to study start; SD = standard deviation; N = total number of participants; n (%) = number (percentage) of participants in a given category.

TABLE 4
Frequency of gE-specific CD4⁺ T cells expressing at least two activation
markers per 10⁶ cells and geometric mean concentrations of anti-gE antibodies

| | | Cell-mediated immunity | | | | Humoral immunity | | |
|---|---|---|---|---|---|---|---|---|
| | | Frequency of CD4²⁺ | | | | Anti-gE antibodies | | |
| Group | Timing | N | Q1 | Median | Q3 | N | GMC | 95% CI |
| HZ-PreVac | Month 0 (pre-vac) | 152 | 1.0 | 67.4 | 138.2 | 204 | 1784.3 | 1572.9-2024.1 |
| | Month 1 (post-D 1) | 177 | 240.6 | 425.1 | 673.0 | 204 | 29959.0 | 26633.6-33699.6 |
| | Month 3 (post D 2) | 170 | 1464.5 | 2312.1 | 4148.3 | 204 | 49327.2 | 45388.2-53608.1 |
| HZ-NonVac | Month 0 (pre-vac) | 140 | 1.0 | 58.1 | 160.3 | 202 | 1408.5 | 1203.3-1648.8 |
| | Month 1 (post-D 1) | 170 | 219.7 | 426.8 | 733.4 | 202 | 25233.7 | 22072.3-28848.0 |
| | Month 3 (post D 2) | 177 | 1448.6 | 2214.2 | 3734.5 | 204 | 51618.5 | 47224.8-56420.9 |

N = number of participants with available results;
HZ-NonVac = participants who never received the live-attenuated zoster vaccine (ZVL);
HZ-PreVac = participants who received ZVL ≥5 years prior to study start;
95% CI = 95% confidence interval;
CD4²⁺, CD4⁺ T cells expressing at least two activation markers among CD40 ligand, interleukin-2, tumor necrosis factor-α, interferon-γ;
GMC, geometric mean concentrations;
Q1, Q3, first and third quartiles;
pre-vac = before first dose of HZ/su;
post D 1 = one month after first dose of HZ/su;
post D 2 = one month after second dose of HZ/su

TABLE 5
Adjusted GMCs and adjusted GMC ratio of anti-gE antibody concentrations
1 month post-dose 2 (According to Protocol Cohort for immunogenicity)

| | | | | | | | | GMC ratio | | |
|---|---|---|---|---|---|---|---|---|---|---|
| | HZ-NonVac | | | | HZ-PreVac | | | (HZ-NonVac/HZ-PreVac) | | |
| | Adjusted | 95% CI | | | Adjusted | 95% CI | | | 95% CI | |
| N | GMC | LL | UL | N | GMC | LL | UL | Value | LL | UL |
| 204 | 50522.9 | 44347.4 | 57558.4 | 204 | 48589.4 | 42649.4 | 55356.6 | 1.04 | 0.92 | 1.17^{†} |

HZ-NonVac = participants who never received the live-attenuated zoster vaccine (ZVL);
HZ-PreVac = participants who received ZVL ≥5 years prior to study start;
Adjusted GMC = geometric mean antibody concentration adjusted for group-matching variable;
N = Number of participants with both pre- and post-vaccination results available;
95% CI = 2-sided 95% Confidence Interval;
LL = lower limit;
UL = upper limit
^{†}= primary objective considered met if ≤1.5

### Example 4—Immunogenicity in Adults with Solid Tumors Vaccinated Before or During Immunosuppressive Chemotherapy Treatment

Example 4 provides the results of a phase II/III, randomized, placebo-controlled, observer-blind, multicentre, clinical trial of GSK Biologicals' VZV gE/AS01B vaccine (HZ/su vaccine, two dose) in adults with solid tumors (ST), with the first dose of vaccine administered either before or during immunosuppressive chemotherapy (NCT ClinicalTrials identifier: NCT01798056).

Methods:

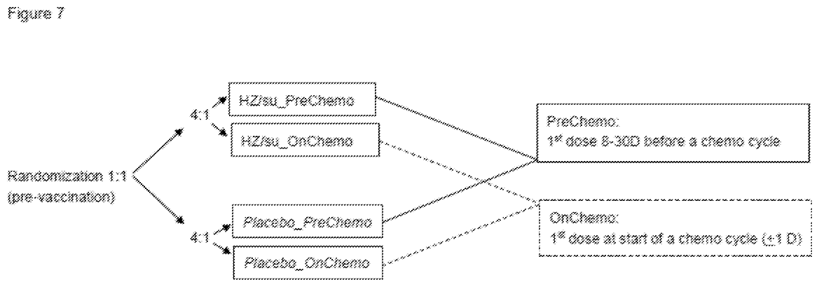

Adults (>18 years of age) with solid tumors (ST) received two doses of either HZ/su or placebo (PI), administered intramuscularly 1-2 months apart. Subjects were randomized 4:4:1:1 to receive the first dose either 8-30 days (D) pre-chemo (HZ/su-PreC group, placebo-PreC), or at the start of chemotherapy (±1 Day (D)) (HZ/su-OnC, PI-OnC). Thus, the ST adults were randomized 1:1 to receive 2 doses of HZ/su or placebo intramuscularly 1-2 months apart, and these two groups (HZ/su and placebo) were further randomized (4:1) as follows: (i) the PreChemo group received the first vaccination 8-30 days before starting a chemo cycle; (ii) the OnChemo group received the first vaccination at the start of a chemo cycle (FIG. 7). All second doses were administered 1-2 months after the first dose and at start (plus/minus one Day) of a subsequent chemo cycle. The HZ/su vaccine contained 50 μg of VZV gE, and AS01B (50 μg 3-O-desacyl-4′-monophosphoryl lipid A (MPL, produced by GSK) and 50 μg QS21, and liposomes).

232 subjects were included in the total vaccinated cohort: 117 HZ/su recipients (90 PreChemo, 27 OnChemo) and 115 placebo recipients (91 PreChemo, 24 OnChemo).

185 subjects were included in the according-to-protocol (ATP) cohort for humoral immunogenicity: 65 HZ/su_PreChemo, 78 Placebo_PreChemo, 22 HZ/su_OnChemo, 20 Placebo_OnChemo.

58 subjects were included in the ATP sub-cohort for CMI: 27 HZ/su_PreChemo, 31 Placebo_PreChemo.

Demographic characteristics were comparable between study groups (Table 6). The most common ST was breast cancer, followed by colorectal, lung and other (including gastric, endometrial, ovarian, head and neck, larynx, mouth, sinus, tonsil, liposarcoma myxoid, liver, oesophageal, renal, sarcoma, stomach, testicular embryonic carcinoma, thyroid, tongue, cervix, urotelial, uterine leiomyosarcoma) types of cancer.

TABLE 6
Demographic characteristics of study subjects
(ATP cohort for humoral immunogenicity)

| | | PreChemo | | | | OnChemo | | | |
|---|---|---|---|---|---|---|---|---|---|
| | | HZ/su | | Placebo | | HZ/su | | Placebo | |
| Characteristics | | N = 65 | | N = 78 | | N = 22 | | N = 20 | |
| Age (years) at dose 1 (mean ± SD) | | 55.5 ± 11.3 | | 56.9 ± 11.0 | | 55.6 ± 10.5 | | 57.2 ± 11.9 | |
| Female (n [%]) | | 42 | (64.6) | 47 | (60.3) | 15 | (68.2) | 13 | (65.0) |
| Solid tumor diagnosis | Bladder | 1 | (1.5) | 1 | (1.3) | 0 | (0.0) | 0 | (0.0) |
| (n [%]) | Breast | 34 | (52.3) | 39 | (50.0) | 13 | (59.1) | 9 | (45.0) |
| | Colorectal | 12 | (18.5) | 20 | (25.6) | 5 | (22.7) | 2 | (10.0) |
| | Lung | 4 | (6.2) | 7 | (9.0) | 0 | (0.0) | 3 | (15.0) |
| | Melanoma | 1 | (1.5) | 0 | (0.0) | 0 | (0.0) | 0 | (0.0) |
| | Pancreas | 1 | (1.5) | 0 | (0.0) | 0 | (0.0) | 1 | (5.0) |
| | Prostate | 2 | (3.1) | 1 | (1.3) | 0 | (0.0) | 1 | (5.0) |
| | Other | 10 | (15.4) | 10 | (12.8) | 4 | (18.2) | 4 | (20.0) |

ATP = according-to-protocol; N = total number of subjects; SD = standard deviation; n (%) = number (percentage) of subjects in a given category.

Immunogenicity Assessment:

Blood samples for immunogenicity assessment were collected and assessed as shown in Table 7. Vaccine response rates (VRRs) and geometric means (GMs)/means were evaluated for gE humoral immune and gE-specific CD4*cell-mediated immune (CMI) responses 1 month (M2) and 12 months (M13) post-dose 2.

Anti-gE humoral immune responses (antibody concentrations and vaccine responses), were determined by enzyme-linked immunosorbent assay (ELISA)) and were assessed in all subjects pre-vaccination through 12 months (M13) post dose 2.

In a sub-cohort of subjects from the PreChemo subgroups (HZ/su-PreC, placebo-PreC), M2 and M13 gE-specific CD4⁺ T-cell mediated immune (CMI) responses were assessed (frequencies and vaccine responses for gE-specific CD4⁺ T-cells expressing ≥2 activation markers from among interferon gamma [IFN-γ], interleukin-2 [IL-2], tumor necrosis factor alpha [TNF-α], and CD40 ligand [CD40L] as determined by intracellular cytokine staining following stimulation with gE peptides).

TABLE 7

| | | | | M 6 | |
|---|---|---|---|---|---|
| | M 0 | M 1 | M 2 | (4-13)* | M 13 |
| Vaccination | First | Second** | | | |
| Blood | Humoral; | Humoral; | Humoral; | Humoral | Humoral; |
| Sampling | CMI*** | CMI*** | CMI*** | | CMI*** |

M = month
First vaccination: administered 8-30 D before a chemotherapy cycle in PreChemo group, administered plus/minus 1 D at start of chemotherapy cycle in OnChemo group;
*start of last chemotherapy cycle;
**the second dose of vaccine was administered 1-2 months after the 1^{st} dose and at the start (plus/minus 1 Day) of a subsequent chemotherapy cycle;
***CMI assessed only in a sub-cohort of the PreChemo groups (HZ/su and Placebo).

TABLE 8
Humoral and cellular immune responses (ATP cohort for humoral
immunogenicity and ATP sub-cohort for CMI, respectively)

| | | | | |
|---|---|---|---|---|
| | HZ/su-PreC | Pl-PreC | HZ/su-OnC | Pl-OnC |
| Time | (HZ/su) | (placebo) | (HZ/su) | (placebo) |
| point | Value | Value | Value | Value |

Humoral immune responses (ATP cohort for humoral immunogenicity)

| | | | | | |
|---|---|---|---|---|---|
| VRR*, | M 2 | 93.8 | 0.0 | 63.6 | 0.0 |
| % (95% CI) | | (85.0-98.3) | (0.0-4.7) | (40.7-82.8) | (0.0-18.5) |
| | | N = 65 | N = 76 | N = 22 | N = 18 |
| | M 13 | 52.9 | 0.0 | 47.1 | 0.0 |
| | | (38.5-67.1) | (0.0-6.5) | (23.0-72.2) | (0.0-23.2) |
| | | N = 51 | N = 55 | N = 17 | N = 14 |
| GMC, | M 2 | 22974.3 | 1120.9 | 9328.0 | 854.6 |
| mIU/ml (95% CI) | | (19080.0-27663.5) | (903.9-1390.0) | (4492.5-19368.2) | (534.1-1367.2) |
| | | N = 65 | N = 78 | N = 22 | N = 20 |
| | M 13 | 4563.0 | 1178.9 | 4229.5 | 708.5 |
| | | (3532.8-5893.7) | (923.3-1505.1) | (2073.8-8626.0) | (376.9-1331.8) |
| | | N = 51 | N = 56 | N = 17 | N = 14 |

| | | | |
|---|---|---|---|
| Adjusted** | M 2 | 23.2 | — |
| GMC ratio | | (17.9-30.0) | |
| (HZ/su:placebo) | | p < 0.0001 | |
| (95% CI) | | | |

CMI responses (ATP sub-cohort for CMI)

| VRR*, | | M 2 | 50.0 | 0.0 | — |
|---|---|---|---|---|---|
| % (95% CI) | | | (28.2-71.8) | (0.0-12.8) | |
| | | | N = 22 | N = 27 | |
| | | M 13 | 17.6 | 0.0 | — |
| | | | (3.8-43.4) | (0.0-20.6) | |
| | | | N = 17 | N = 16 | |
| Freq., | GM | M 2 | 781.8 | 78.7 | — |
| | 95% CI | | (535.2-1110.4) | (13.7-162.9) | |
| | | | N = 22 | N = 27 | |
| | mean | M 13 | 523.83 | 125.78 | — |
| | | | N = 18 | N = 19 | |

| | | | |
|---|---|---|---|
| Adjusted** GM | M 2 | 9.94 | — |
| frequency ratio | | (3.63-27.19) | |
| (HZ/su:placebo) | | p < 0.0001 | |
| (95% CI) | | | |

HZ/su-PreC: first of 2 HZ/su vaccinations at 8-30 days prior to the start of a chemotherapy cycle;
Pl-PreC: first of 2 placebo administrations at 8-30 days prior to the start of a chemotherapy cycle;
HZ/su-OnC: first of 2 HZ/su vaccinations at the start of a chemotherapy cycle (±1 day);
Pl-OnC: first of 2 placebo administrations at the start of a chemotherapy cycle (±1 day);
N: number of subjects with available results;
VRR: vaccine response rate;
GM: geometric mean;
GMC: geometric mean anti-gE antibody ELISA concentrations;
Freq.: frequency of gE-specific CD4[2+] T-cells (per 10⁶ total CD4+ T-cells); %, percentage of subjects;
CI: confidence interval
IU: international unit;
M 2: Month 2 (1 month post-dose 2): M 13, Month 13 (12 months post-dose 2).
In Table 8, the p-value is relative to the null hypothesis H₀: HZ/su:placebo = 1. Bolded values indicate immunogenicity success criteria of the primary objective (lower limit [LL] of 95% CI for GMC HZ/su:placebo ratio ≥60% - humoral immunogenicity) and secondary objectives (LL of 95% CI for VRR ≥3 - humoral immunogenicity and for GM frequency HZ/su: placebo ratio ≥1 - CMI) that were met.
Humoral VRR is the percentage of subjects with a vaccine response as follows: for initially seronegative subjects (anti-gE antibody concentration below the cut-off [97 mIU/ml]), at least a 2-fold increase as compared to the cut-off; for initially seropositive subjects (anti-gE antibody concentration above the cut-off), at least a 4-fold increase as compared to the pre-vaccination antibody concentration. CMI VRR is the percentage of subjects with vaccine response: for subjects with pre-vaccination T-cell frequencies below the threshold (320 gE-specific CD4[2+] T-cells/10⁶ CD4+ T-cells), at least a 2-fold increase as compared to the threshold; for subjects with pre-vaccination T-cell frequencies above the threshold, at least a 2-fold increase as compared to pre-vaccination T-cell frequencies. **, adjusted for baseline values.

Anti-gE Humoral Immune Responses:

The following HZ/su immunogenicity success criteria for M2 were met (Table 8):
The lower limit (LL) of the 95% confidence interval (CI) of geometric mean concentration (GMC) HZ/su:placebo ratio for anti-gE antibody concentrations was 17.9 in the PreChemo group (>3).
The LL of the 95% CI for humoral vaccine response rates (VRR) was 85.0% (≥60%).
The LL of the 95% CI for CMI geometric mean (GM) frequency HZ/su:placebo ratio of gE-specific CD4+ T-cell frequencies was 3.63 (>1).

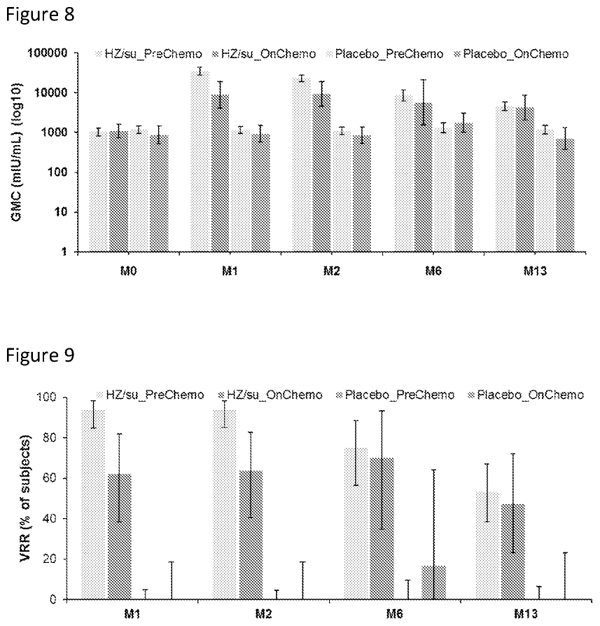

M1 through M13 anti-gE GMC were higher for HZ/su than for the corresponding placebo groups (except for OnChemo recipients at M6, for which responses to HZ/su and placebo were similarly high) (FIG. 8). 47.1% (M13)-93.8% (M1, M2) of HZ/su subjects met the criteria for humoral vaccine response, compared to 0.0%-16.7% for the placebo groups (FIG. 9).

Both GMC and VRR tended to decrease with time in HZ/su recipients; however, GMC values remained higher than before vaccination in the HZ/su subgroups.

1 month post-dose 1, HZ/su recipients in the PreChemo subgroup had higher immune responses compared to the HZ/su_OnChemo subgroup. No differences were observed at M13 (FIG. 8 & FIG. 9).

gE-specific CMI Responses:

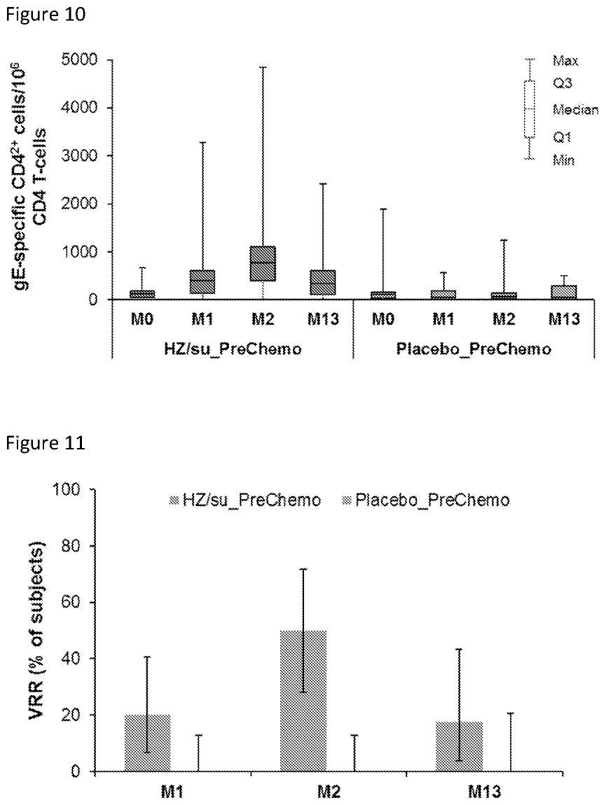

M2 and M13 mean frequencies of gE-specific CD4²⁺ T-cells values were significantly higher in the HZ/su_PreChemo than in the Placebo_PreChemo group. The mean frequencies for HZ/su recipients peaked at M2 (FIG. 10).

In the HZ/su PreChemo group, 17.6% (M13)-50.0% (M2) met the criteria for CMI vaccine response, compared to 0.0% for the Placebo_PreChemo group (FIG. 11).

Safety Assessment: Solicited adverse events (AE) were recorded for 7 days and unsolicited AE and medically-attended AE (MAE) for 30 days after each dose. Potential immune-mediated diseases (pIMD) and serious AE (SAE) were recorded until study end. Most solicited general AE were reported at comparable frequencies by HZ/su and placebo recipients (data not shown). A high background incidence was reported in the placebo groups (65.1% Placebo_PreChemo, 70.8% Placebo_OnChemo). The frequency of solicited local AE was higher in the HZ/su groups than in the corresponding placebo groups. Pain and fatigue were the most commonly reported solicited AE. Unsolicited AE, MAE and SAE were reported at similar frequencies among HZ/su and placebo recipients. One pIMD (Placebo_OnChemo) and 23 fatal SAE were reported. No SAE was considered as vaccine-related by the investigators. No differences in safety outcomes were observed between the HZ/su_OnChemo and the HZ/su_PreChemo groups. Suspected HZ cases were reported for 1 subject in the HZ/su_PreChemo group at M1, and 2 subjects in the Placebo_PreChemo group at M6 and M13, respectively.

Results:

185 subjects (65 HZ/su-PreC, 78 PI-PreC, 22 HZ/su-OnC, 20 PI-OnC) were included in the according-to-protocol (ATP) cohort for humoral immunogenicity and 58 (27 HZ/su-PreC, 31 PI-PreC) in the ATP sub-cohort for CMI. The most common ST were breast tumors (54% HZ/su, 49% placebo), followed by colorectal, lung, then other. Humoral and CMI VRRs were higher in HZ/su than PI groups at M2 and M13. GM concentration (GMC) was highest at M2 in HZ/su-PreC. M13 GMCs were similar in the HZ/su-PreC and HZ/su-OnC groups.

Conclusion:

The present results indicate the HZ/su vaccine was immunogenic (as measured up to M13) in ST adults receiving immunosuppressive chemotherapy, with the first vaccine dose administered either before or at the start of a chemotherapy cycle.

### Example 5: Immunogenicity and Safety of HZ/Su in Adults Post Renal Transplant

Solid organ transplants (SOT) recipients are at increased risk for Herpes Zoster (HZ) infections due to their daily immunosuppressive therapy for the prevention of host-versus-allograft rejection (Insinga et al., J Gen Intern Med 2005; 20:748-53) with a ^{˜}7-fold larger incidence with respect to the overall United States population, and a 17-32% HZ incidence rate during the first 4 years post-transplant (Pergam et al., Transpl Infect Dis 2011; 13:15-23).

The present study was performed to assess immunogenicity and safety of HZ/su in adult renal transplant (RT) recipients (RTR) on chronic immunosuppressive therapy (calcineurin inhibitor or sirolimus (CIS); corticosteroids (CS); and/or mycophenolate compound (MC). RT was chosen as it can be representative of SOTs due to the nature of administered immunosuppressive therapies. GSK's HZ subunit candidate vaccine, HZ/su, was administered; HZ/su contains 50 μg of VZV gE and the adjuvant AS01B (50 μg 3-O-desacyl-4′-monophosphoryl lipid A (MPL, produced by GSK), 50 μg Quillaja saponaria Molina, fraction 21 (QS-21), and liposomes).

Methods: In this phase III, observer-blind, multicenter study (NCT02058589), RTRs≥18 YOA were randomized 1:1 to receive two doses of either HZ/su or placebo, administered intramuscularly 1-2 months apart. Subjects were also stratified by age (18-49; ≥50 years) and by immunosuppressive therapy. Blood samples are taken at M0, M1, M2, M4, M7, M10 and M13; results available to M2 are presented here.

The gE-specific vaccine response rates (VRRs) and geometric means (GMs) were assessed for humoral and CD4+ cell-mediated immune (CMI) responses 1 month post dose 2 (M2). Solicited adverse events (AEs) were recorded for 7 days and unsolicited AEs and medically-attended AEs (MAEs) for 30 days after each dose. Solicited general and unsolicited AEs were also collected for 7 days prior to dose 1. Potential immune-mediated diseases (pIMD) and serious AE (SAE) are recorded until 1 year post dose 2

Immunogenicity Assessment: Anti-gE humoral immune responses (antibody [Ab] concentrations and vaccine responses, as determined by enzyme-linked immunosorbent assay [ELISA]) were assessed in all subjects. gE-specific CD4+ T cell mediated immune responses (CMI) (frequencies and vaccine responses for gE-specific CD4+ T cells expressing 2 activation markers from among interferon gamma [IFN-γ], interleukin-2 [IL-2], tumor necrosis factor alpha [TNF-α] and CD40 ligand [CD40L] as determined by intracellular cytokine staining following stimulation with gE peptides) were assessed in a subset of subjects.

Study Participants: Demographic characteristics were comparable between HZ/su and placebo recipients (data not shown). Of the 264 vaccinated subjects (132 in HZ/su group and 132 in placebo), 240 (121 HZ/su; 119 placebo) and 72 (36 in each group) were included in the M2 according-to-protocol cohorts for humoral immunogenicity and CMI, respectively.

Anti-gE humoral immune responses: All HZ/su immunogenicity success criteria for M2 were met (Table 9):
The lower limit (LL) of the 95% confidence interval (CI) of the vaccine response rates (VRR) for anti-gE Ab concentrations was 71.9% (≥60%).
The LL of the 95% CI of the geometric mean (GM) ratio (HZ/su over placebo) of anti-gE Ab concentrations was 10.90 (>3).
The LL of the 95% CI of the VRR for gE-specific CD4+ T cell frequencies was 51.3% (≥50%).
The LL of the 95% CI of the GM ratio (HZ/su over placebo) of gE-specific CD4+ T cell frequencies was 5.92 (>1).

TABLE 9
VRR, GM and GM ratios for anti-gE antibody ELISA concentrations
and gE-specific CD4+ T cell frequencies at M 2 (ATP
cohorts for humoral immunogenicity and CMI, respectively)

| HZ/su | | Placebo | | |
|---|---|---|---|---|
| | Value | | Value | Adjusted ratio |
| N | (95% CI) | N | (95% CI) | HZ/su:placebo |

Humoral immune response (anti-gE antibody ELISA concentration)

| | | | | | |
|---|---|---|---|---|---|
| VRR, % | 121 | 80.2 | 119 | 4.2 | — |
| | | (71.9; 86.9) | | (1.4; 9.5) | |
| Adjusted* GMC, | 121 | 19983.3 | 119 | 1427.3 | 14.00 |
| mIU/ml | | (15779.7; 25306.7) | | (1310.0; 1555.2) | (10.90; 17.99) |
| | | | | | p < 0.0001 |

CMI response (gE-specific CD4⁺ T cell frequencies)**

| | | | | | |
|---|---|---|---|---|---|
| VRR, % | 28 | 71.4 | 28 | 0.0 | — |
| | | (51.3; 86.8) | | (0.0; 12.3) | |
| Adjusted* GM, | 28 | 1440.5 | 28 | 83.5 | 17.26 |
| events/10⁶ CD4⁺ T | | (1044.4; 1959.6) | | (8.6; 181.5) | (5.92; 50.36) |
| cells | | | | | p < 0.0001 |

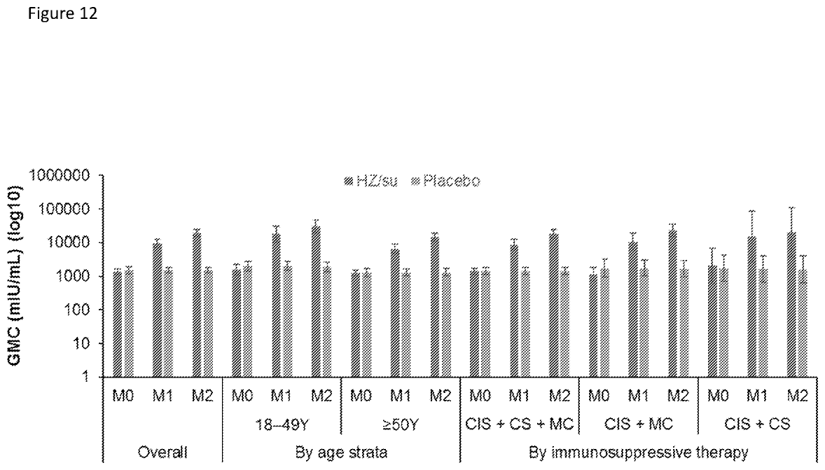

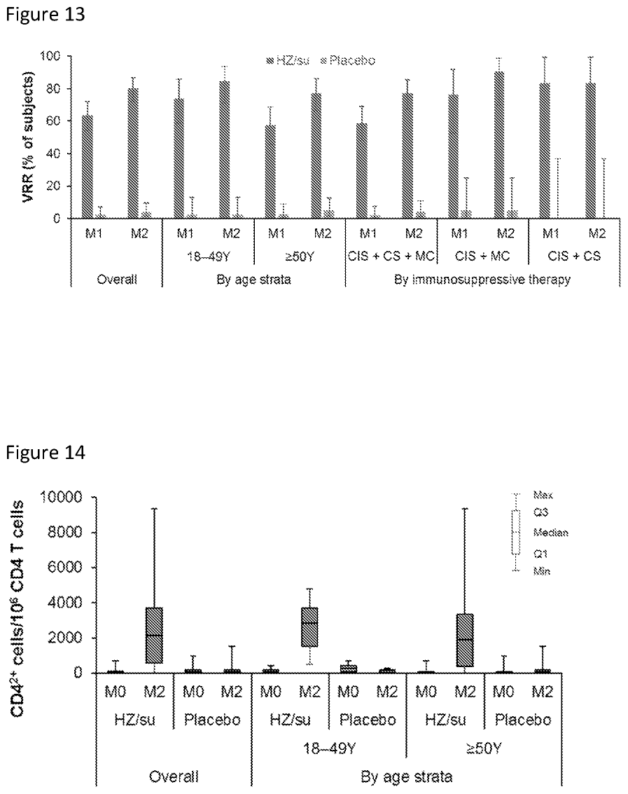

VRR = vaccine response rate#; GM(C) = geometric mean (concentration); M 2 = month 2 (1 month after last vaccination); ATP = according-to-protocol; N = number of subjects with available results; CI = confidence interval; IU = international units.
*adjusted for baseline values; **for the inferential analysis, the frequency of CD4+ T cells producing ≥2 activation markers (IFN-γ, IL2, TNFα, and CD40 Ligand) upon in vitro stimulation with the antigen (induction condition) is calculated, by adding an offset of 0.5 to the number of activated CD4+ T cells (numerator) divided by the total number of CD4+ T cells involved (denominator).
#VRR: (i) for humoral immune response: (a) in initially seronegative subjects, the post-vaccination antibody concentration ≥4-fold the cut-off for anti-glycoprotein E (gE) (4 × 97 mIU/ml); (b) in initially seropositive subjects, the post-vaccination antibody concentration ≥4-fold the pre-vaccination antibody concentration; (ii) for cell-mediated immunogenicity (CMI): (a) in subjects with initial pre-vaccination T cell frequencies below the cut-off (320/106 CD4+ T cells), the post-vaccination T cell frequencies ≥2-fold the cut-off (2 × 320/106 CD4+ T cells); (b) in subjects with initial pre-vaccination T cell frequencies above the cut-off, the post-vaccination T cell frequencies ≥2-fold the pre-vaccination T cell frequencies.
Bolded values indicate that immunogenicity success criteria of primary objective (lower limit of 95% CI ≥60% for VRR - humoral) and secondary objectives (lower limit of 95% CI ≥50% for VRR - CMI, >3 for GM ratio - humoral, >1 for GM ratio - CMI) were met.
One month post-dose 2, anti-gE geometric mean concentrations (GMC) were significantly higher in HZ/su compared to placebo recipients (FIG. 12). GMC were high in both HZ/su age cohorts, but tended to be higher in the younger age cohort; GMC were similar for the different immunosuppressive therapy strata.
Most HZ/su recipients (≥77.2%) met the criteria for humoral vaccine response at M 2 (FIG. 13); VRR tended to be higher in the younger age group compared to the older age group, but were similar among the different immunosuppressive therapy subgroups.

gE Specific Cell Mediated Immunity (CMI) Responses:

The median frequency of gE-specific CD42+ T cells 1 month post-dose 2 was significantly higher in HZ/su versus placebo recipients and tended to be higher in HZ/su recipients 18-49 years old compared to those ≥50 years old (FIG. 14).

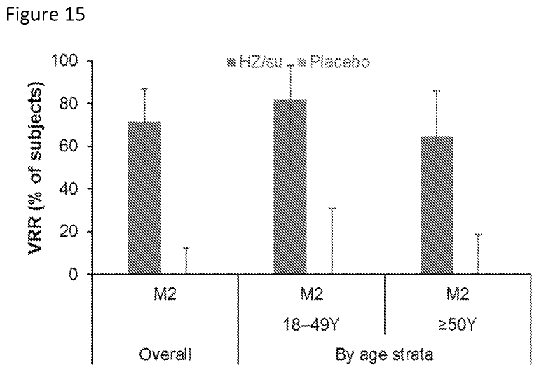

One month post-dose 2, ≥64.7% of subjects in the HZ/su group met the criteria for CMI vaccine response compared to none in the placebo group (FIG. 15). Among HZ/su recipients, VRR tended to be higher in the younger age group compared to the older age group, although the difference was not significant.

Safety: Solicited general AE, unsolicited AE, MAE and SAE were reported at similar frequencies in both groups (data not shown). A high background incidence was recorded for solicited general AE in placebo recipients (55.3%). The frequency of solicited local AE was higher in HZ/su vs placebo groups. No pIMD, vaccine-related SAE or transplant rejections were reported.

Results and Conclusion: At M2, 240 subjects (121 HZ/su; 119 placebo) were included in the humoral immunogenicity according-to-protocol (ATP) cohort. All immunogenicity success criteria were met at M2. VRRs for ATP humoral immune cohort and CMI sub-cohort (72 subjects: 36 HZ/su; 36 placebo) were higher in HZ/su groups. Humoral GM concentrations and CMI GM frequencies were significantly higher in HZ/su compared to placebo groups. HZ/su was immunogenic in adults with RT at M2.

1.-26. (canceled)

27. A method for prevention of herpes zoster (HZ) in a human subject comprising the step of administering to said human subject one dose of an immunogenic composition comprising a Varicella Zoster Virus (VZV) gE antigen truncated to remove the carboxy terminal anchor region, in combination with an adjuvant comprising a saponin, a TLR-4 agonist and liposomes.

28-37. (canceled)

38. The method according to claim 27, wherein the human subject is 50 years of age or older.

39. The method according to claim 27, wherein the human subject is 70 years of age or older.

40. The method according to claim 27, wherein the human subject received a live-attenuated VZV vaccine at least 3 years earlier.

41. The method according to claim 27, wherein the VZV gE antigen is not in the form of a fusion protein.

42. The method according to claim 27, wherein the VZV gE antigen comprises the sequence of SEQ ID NO: 1.

43. The method according to claim 27, wherein the VZV gE antigen is present in an amount of between 20 to 100 μg per dose.

44. The method according to claim 27, wherein the VZV antigen is present in an amount of 50 μg per dose.

45. The method according to claim 27, wherein the saponin is QS21.

46. The method according to claim 45, wherein the QS21 is present in an amount of 1 to 100 μg per dose.

47. The method according to claim 45, wherein the QS21 is present in an amount of 50 μg per dose.

48. The method according to claim 27, wherein the TLR-4 agonist is 3-O-desacyl-4′-Monophosphoryl Lipid A (3D-MPL).

49. The method according to claim 48, wherein the 3D-MPL is present in an amount of at least 25 μg per dose.

50. The method according to claim 27, wherein the liposomes further comprise a sterol.

51. The method according to claim 27, wherein the liposomes comprise dioleoyl phosphatidylcholine (DOPC) and cholesterol.

52. The method according to claim 27, wherein the immunogenic composition further comprises an additional VZV antigen selected from live attenuated VZV OKA strain and killed VZV OKA strain.

53. A method for prevention of herpes zoster in a human individual scheduled to receive immunosuppressive medical therapy, comprising administering prior to the start of said immunosuppressive therapy a single dose of an immunogenic composition comprising a VZV gE antigen truncated to remove the carboxy terminal anchor region, in combination with an adjuvant comprising a saponin, a TLR-4 agonist and liposomes.

54. The method according to claim 53 wherein said scheduled immunosuppressive therapy is selected from chemotherapy, radiation therapy, or immunosuppressive pharmaceutical compounds.

55. The method according to claim 53 wherein said single dose is administered at least thirty days prior to the start of immunosuppressive medical therapy.

56. The method according to claim 53 wherein said subject received a live-attenuated VZV vaccine at least 3 years earlier.
